(19) United States
(12) Patent Application Publication
(10) Pub. No.: US 2012/0021012 A1
(43) Pub. Date: Jan. 26, 2012

(54) NOVEL PHARMACEUTICAL USES FOR NANOPARTICLES

(75) Inventors: Volodymyr Lysenko, Villeurbanne (FR); Alain Geloen, Lyon (FR); Barbara Mognetti, Turin (IT)

(73) Assignees: CENTRE NATIONAL DE LA RECHERCHE SCIENTIFIQUE (C.N.R.S), Paris (FR); INSTITUT NATIONAL DE LA SANTE ET DE LA RECHERCHE MEDICALE (INSERM), Paris (FR); UNIVERSITE CLAUDE BERNARD DE LYON (UCBL), VILLEURBANNE CEDEX (FR)

(21) Appl. No.: 13/124,650

(22) PCT Filed: Oct. 16, 2009
(86) PCT No.: PCT/FR2009/051988

§ 371 Date: Oct. 6, 2011

(30) Foreign Application Priority Data
Oct. 17, 2008 (FR) 0857100

(51) Int. Cl. A61K 9/14; A61K 33/00; A61P 35/00; A61P 35/04; B82Y 5/00

(52) U.S. Cl. 424/400; 424/600; 977/773; 977/915

(57) ABSTRACT

The present invention relates to SiC nanoparticles to be used in the context of cancer treatment, said nanoparticles preferably having a size less than 100 nm.

The subject of the present invention concerns novel pharmaceutical uses of nanoparticles, in particular in the field of cancer treatment.

Cancer cells can be eliminated by chemotherapy using synthetic molecules, mainly antimitotics. Conventional antineoplastic drugs inhibit cell proliferation by interacting non-specifically with the processes of cell replication (duplication of nucleic acids, correct functioning of the mitotic spindle, synthesis of purines and pyrimidines, etc.). On the other hand, innovative antineoplastic drugs are chiefly monoclonal antibodies directed against specific tumour antigens. These molecules may be used alone (e.g. Herceptin®) or they may convey cytotoxic drugs or radioactive particles (for example Ibritumomab administered in combination with other antineoplastic drugs to treat non-Hodgkin's lymphomas).

The use of antimitotic molecules has a major drawback. These molecules effectively attack all the cancer cells, but also non-cancerous cells. This means that numerous types of cells which divide rapidly are also affected (skin, red blood cells, white blood cells, platelets . . . ). The side effects can therefore be quite considerable, and reduced therapeutic schedules are frequently observed to limit the efficacy of the treatment.

In addition, some conventional antineoplastic drugs are carcinogenic, which implies the risk of developing a new tumour in patients given this treatment but also in persons in charge of handling drugs. Second generation drugs which in most cases are still considered to be second choice treatments, together with minor side effects, often lead to a less effective therapeutic result than with conventional drugs, and in addition they are extremely costly.

It is therefore an objective of the present invention to provide a novel anticancer drug that is selective and efficient i.e. a drug efficient against cancer cells but which does not attack non-cancer cells, and whose production is also relatively low cost.

The objective of the present invention is to provide an anticancer drug inhibiting the cell survival of cancer cells, without inhibiting the survival of non-cancer cells.

The present invention pertains to SiC nanoparticles (silicon carbide) for use thereof in the treatment of cancer.

The present invention therefore concerns the use of SiC nanoparticles of size less than 100 nm for preparing a drug intended to treat cancer.

SiC nanoparticles are chemically, thermally and mechanically stable. Additionally, they have stability against radiation (UVA, UVB and X-rays in particular). They are non-toxic and easily incorporated, in particular into healthy living cells, and they can therefore be used as fluorescent marker (Botsoa et al., Applied Physics Letters, 92, 173902, 2008).

The inventors have found, after incorporating SiC nanoparticles, that such particles have a strong therapeutic effect on cancer cells, without however affecting non-cancer cells.

Indeed, the SiC nanoparticles used under the present invention cause the death of human cancer cells whilst allowing healthy human cells to survive.

According to one preferred embodiment, the above-mentioned nanoparticles have a size of between 0.5 nm and 1 μm.

According to one particularly preferred embodiment, the size of said nanoparticles is less than 10 nm, preferably less than 7 nm and more preferably less than 5 nm.

According to one advantageous embodiment, the present invention concerns nanoparticles such as defined above for use thereof in the treatment of cancer, characterized in that they are in the form of a suspension.

Preferably, the nanoparticles according to the present invention are in the form of a suspension in an aqueous physiological buffer.

The expression “aqueous physiological buffer” herein designates any compatible physiological solution which can be administered to a human being.

Among the suitable physiological buffers, particular mention may be made of physiological saline solution, in vitro culture media for cells such as the RPMI and DMEM media for example.

Under the present invention, said nanoparticles in suspension form are present at a concentration ranging from 0 to 20 g/L in said suspension.

According to one particular embodiment, the above-mentioned nanoparticles are present in said suspension at a concentration of between 0.5 g/L and 2 g/L.

The present invention also concerns the above-mentioned SiC nanoparticles for use thereof in the treatment of cancer, characterized in that these nanoparticles are administered via intravenous route, for example in the form of an aqueous solution.

The above-mentioned nanoparticles can also be administered via local injection using a syringe.

Another suitable route for their administration is the cutaneous route, for example by applying a cream or lotion containing these nanoparticles to the cancerous site.

It is also possible to envisage administering said nanoparticles via oral route, by swallowing tablets or solutions in which they are contained

According to one preferred embodiment, the nanoparticles used for the present invention are able to be obtained using a SiC substrate etching method, etching being conducted by electrochemical attack of the SiC substrate.

The expression “SiC substrate”, under the present invention, designates any chemical compound comprising silicon atoms (Si) and carbon (C) atoms chemically bonded together. The atomic structure of the SiC substrate (and hence of the nanoparticles) may be single-crystalline, poly-crystalline or amorphous. For example, the polytype of the SiC substrate may be: 6H—SiC, 4H—SiC, 3C—SiC, 15R—SiC, 21R—SiC, 24R—SiC, 27R—SiC, etc. The chemical composition of the SiC substrate for example may be: SiC or any other compound expressed by the formula SixC(1−x) where 0<x<1. The SiC substrate may contain other chemical impurities (e.g. hydrogen, oxygen, nitrogen, aluminium, phosphorus, boron, beryllium, gallium, titanium, chromium) whose concentration (for each impurity) does not exceed 1% (i.e. less than 1%) of the concentration of the Si and C atoms.

Advantageously, the nanoparticles are obtained by etching a bulk SiC substrate.

Under the present invention, the expression SiC “bulk substrate” designates any chemical compound formed of silicon atoms (Si) and carbon (C) atoms chemically bonded together, and of which at least one linear dimension (height, width, length, diameter, etc.) is greater than 1 mm.

Preferably, the etching of the SiC substrate is obtained by electrochemical attack of the SiC substrate. For example, etching is obtained by electrochemical attack during which the SiC substrate is in contact with an electrolyte formed at least of an acid (hydrofluoric acid for example). An electric current passes through this substrate. The etching parameters such as current density, chemical composition, electrolyte concentration, pressure and ambient temperature will be chosen in relation to needs (etching speed, etc).

According to another preferred embodiment, the nanoparticles used for the present invention are able to be obtained using a method for laser ablation of a SiC substrate under a volume of water or other solvent.

This method consists of directing a pulsed laser beam onto a target (here a SiC substrate) which is in contact with a solvent. The impact of the beam causes dispersion of the SiC substrate in the solvent into small nanoparticles. This method has the advantage of not requiring the use of hydrofluoric acid. In addition, it is not necessary to rinse the sample (it is pointed out that when rinsing a large quantity of formed nanoparticles is lost).

According to another preferred embodiment, the nanoparticles used for the present invention are able to be obtained using a laser pyrolysis method, from a mixture of C₂H₂ and SiH₄ for example. These nanoparticles are prepared as described in particular in the following articles: Leconte et al. (2007) J. Anal. Appl. Pyrolysis, 79, 465 and Herlin-Boime et al. (2004) J. Nanopart. Res., 6, 63.

Preferably, the present invention concerns the above-mentioned SiC nanoparticles for use thereof in the treatment of advanced forms of cancer with metastasis.

Any type of cancer can be treated with the nanoparticles, either via intravenous administration or via local application. Particular mention may be made of mouth or throat cancers.

Finally, the present invention also concerns the above-mentioned SiC nanoparticles for use thereof in the treatment of cancer, characterized in that they are administered in combination with another anticancer drug.

The present invention therefore also concerns the above-mentioned SiC nanoparticles in combination with an anticancer drug for use thereof in the treatment of cancer.

Among the anticancer agents, mention may be made of DNA interfering drugs, antimetabolites, enzyme inhibitors, cytokines and drugs interfering with the mitotic spindle.

Among the DNA interfering drugs, mention may be made of alkylating agents such as chlomethine, intercalating agents such as daunorubicin, the inhibitors of topoisomerase I and II such as irinotecan, topotecan or etoposide, the electrophilic intermediates such as the platinum derivatives e.g. cisplatin carboplatin and oxaliplatin.

Among the antimetabolites, particular mention may be made of 5-fluoro-uracil or mercaptopurine.

Among the enzyme inhibitors particular mention may be made of thymidylate synthase inhibitors e.g. raltitrexed, of ribonucleide-diphosphate reductase inhibitors e.g. hydroxyurea, or of dihydrofolate reductase inhibitors e.g. methotrexate.

Among the cytokines, interferon alpha may be particularly cited.

Among the drugs interfering with the mitotic spindle, particular mention may be made of vinblastine, vincristine, vindesine, navelbine or the taxanes such as taxol, paclitaxel or docetaxel.

As anticancer agents the following chemotherapy agents may also be cited: azathioprine, capecitabine, cytarabine, floxuridine, fludarabine, gemcitabine, methotrexate, pemetrexed, busulfan, chlorambucil, cyclophosphamide, ifosfamide, melphalan, mechlorethamine, uramustine, bleomycin, doxorubicin, epirubicin, idarubicin, mitomycin C and mitoxantrone.

It has been shown that the SiC nanoparticles of the present invention have effects that are comparable to those of molecules conventionally used as anticancer agents in terms of efficacy.

In addition, under the present invention it was found that the combination of the nanoparticles according to the invention with a conventional anticancer agent exhibits synergy with regard to therapeutic effect. It is therefore possible to replace conventional treatment with an anticancer agent by a treatment of “cocktail” type comprising an anticancer agent and nanoparticles of the invention. The use of said “cocktail” for the treatment of a cancer in the early stages of development allows the concentration of the conventional anticancer agent to be reduced relative to the concentration of SiC nanoparticles in the “cocktail”, which leads to obtaining an identical therapeutic effect (to the effect obtained with the concentration of anticancer agent used in currently known conventional treatments) but with a reduction in the side effects following from use of the conventional anticancer agent. On the other hand, for the treatment of cancers at a well advanced stage and by maintaining the concentration of anticancer agent at the level used in conventional treatments, the use of said “cocktail” enables the therapeutic effect to be increased.

The present invention also concerns the above-mentioned SiC nanoparticles for use thereof in the treatment of cancer, characterized in that said nanoparticles are chemically functionalized. The expression “functionalized nanoparticles” designates chemically modified nanoparticles i.e. whose surface has been modified, for example by grafting suitable chemical groups. Therefore, the above-mentioned SiC nanoparticles can be subjected to chemical functionalization to modify their surface chemistry, as described in detail in particular in Alekseev et al. (Chemistry of Materials, 2007, vol. 19, 2189-2194). With this functionalization it is possible to address the nanoparticles at specific sites of the cells.

The present invention also concerns the above-mentioned nanoparticles for use thereof in the treatment of cancer, characterized in that at least one anticancer drug is grafted on the surface of said nanoparticles.

The nanoparticles thus modified allow the addressing of anticancer drugs towards the nuclei of cancer cells, and hence allows an improvement in the efficacy of treatment insofar as the SiC nanoparticles used for the present invention, once incorporated in living cells, penetrate as far as the nucleus of said cells (Botsoa et al., Applied Physics Letters, 92, 173902, 2008).

### FIGURES

FIGS. 1, 2 and 3 show the percentage cell survival of different cell lines treated with different concentrations of SiC nanoparticles, after 24, 48 or 72 hours' incubation respectively. The hatched rectangles relate to the results for the HSC cell line (human cancer line); the black rectangles relate to the results for the AT84 cell line (murine cancer line) and the white rectangles relate to the results for the SG cell line (immortalised healthy human cell line).

FIG. 4 illustrates the effect of an anticancer molecule, namely paclitaxel, on different human cell lines after 72 hours' treatment. The percentage cell survival is indicated for different concentrations of paclitaxel. The hatched rectangles relate to the results for the HSC cell line, the black rectangles relate to the results for the AT84 cell line and the white rectangles relate to the results for the SG cell line.

FIG. 5 illustrates the absorption spectrum for solutions of SiC nanoparticles obtained by laser ablation according to Example 2 (absorption in arbitrary units as a function of energy in eV). The vertical dotted line represents the value of E_{g} of the initial SiC substrate.

FIG. 6 illustrates the photoluminescence spectra of solutions of SiC nanoparticles in deionised water, obtained with 3 laser powers (ablation) (photoluminescence signal in arbitrary units as a function of energy in eV) (T=300K and Eₑₓ=5.08 eV). The curve with the squares corresponds to the spectrum of deionised water alone (without nanoparticles), the curve with the circles corresponds to a laser power of 115 mW, the curve with the triangles corresponds to a laser power of 230 mW and the curve with the diamonds corresponds to a laser power of 460 mW. The vertical dotted line represents the E_{g} value of the initial SiC substrate.

FIG. 7 illustrates the effects of the nanoparticles of the present invention (shaded rectangles), of cisplatin (white rectangles) and of the combination of these nanoparticles with cisplatin (hatched rectangles) on different human cell lines after 72 hours' treatment. The three rectangles on the left correspond to the results for the SG cell line; the three rectangles in the middle correspond to the results for the AT84 cell line; and the three rectangles on the right correspond to the results for the HSC cell line.

### EXPERIMENTAL PART

### Example 1

### Preparation of Nanoparticles by Electrochemical Etching and Properties of the Nanoparticles Thus Obtained

### Description of the Assays Conducted

The nanoparticles of silicon carbide (SiC) enter into animal cells. They first attach to elements present in the cytoplasm of cells and then concentrate in the nucleus of the cells.

The effect of the exposure of several cell types to different concentrations of SiC nanoparticles was assayed.

The nanoparticles were produced by electrochemical anodisation of a 3C—SiC polycrystalline substrate with low resistivity and radius of 1 cm. The etching process lasted three hours under UV illumination, at a current intensity of 25 mA/cm² using a 1:1 dilution HF 50% ethanol as electrolyte. After etching, a highly porous network of interconnected nanocrystals of 3C—SiC is formed. The ultra-porous layer is dried naturally in ambient air. The nano-powder of 3C—SiC obtained by grinding the nanoporous layer is placed in suspension in a Krebs buffer solution. This suspension is centrifuged at 5000×g for three minutes to sediment the large insoluble crystals. The upper part containing the very small nanoparticles (smaller than 10 nm) in uniform suspension was used for the assays.

Cell survival was measured using a colorimetric method with 3-(4,5-dimethylthiazol-2-yl)-2,5-diphenyltetrazolium bromide (MTT). This molecule of yellow colour is reduced by mitochondrial dehydrogenase enzymes to a blue-coloured salt. This allows cell viability to be quantified in response to a treatment. The higher the number of cells, the more they convert MTT and hence the more intense the blue colouring.

Cell survival was measured before and after exposure to different concentrations of SiC nanoparticles (0.1-0.5-1 mg/mL in a suspension of RPMI medium 10% FCS).

The cells were cultured in 96-well plates in the presence of SiC nanoparticles for 18 hours after which the media were replaced by standard culture media without any nanoparticles (RPMI medium 10% FCS, Sigma).

Cell viability was measured after different times (24, 48 and 72 hours). The cell cultures were then used to perform the MTT assay.

Cell viability was measured by the difference in optical density at wavelengths of 450 and 504 nm, using a spectrophotometer connected to a computer system (Power Wave, Bio-Tek Instrument Inc.). The measured absorbency is then directly proportional to the number of living cells. The results are expressed as percentage survival measured under the initial conditions before exposure of the cells to the SiC nanoparticles.

### Results

The experiments were performed on three cell lines:
a human cancer cell line (HSC) (human squamous carcinoma cell line HSC-2 taken from the oral cavity);
an immortalised healthy human cell line (SG) (immortalised human cell line of gingival epithelium, donated by Dr. Babich); and
a mouse cancer cell line (AT84) (cell line from spontaneous oral squamous carcinoma of C3H mice, donated by Dr. Shilitoe).

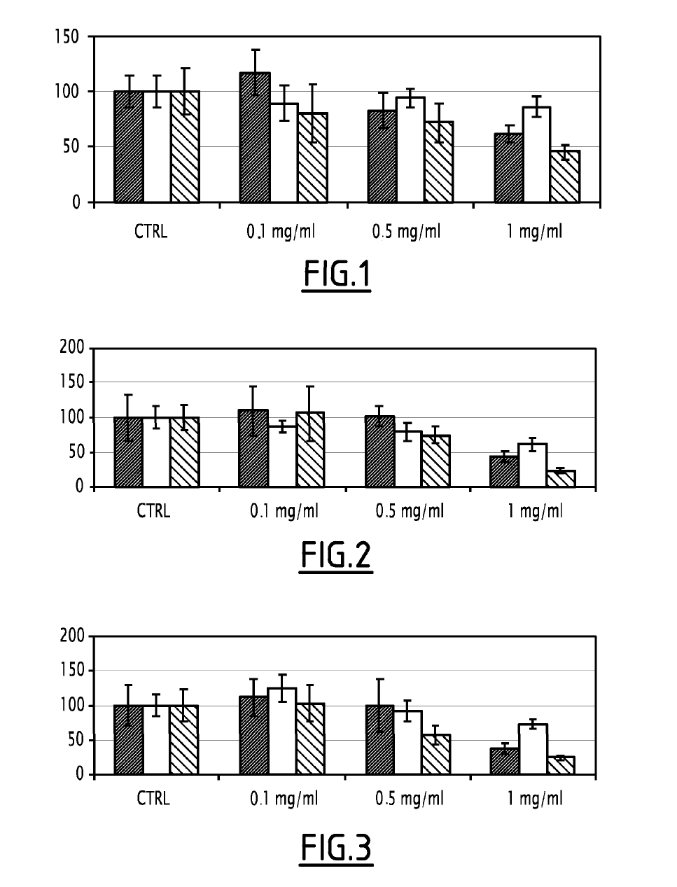

Cell viability was measured after different times (24, 48 and 72 hours). The corresponding results are respectively indicated in FIGS. 1, 2 and 3.

On observing the results it is noted, when comparing the HSC (human cancer) and SG (immortalised human) lines, that the survival of the human cancer cells is significantly reduced on and after 24 hours at a dose of 0.5 mg/mL. This significant effect is only found in the cells of cancer lines.

At a higher concentration, the inhibitory effect of the nanoparticles increases. At 1 mg/mL the viability of the immortalised human cells is slightly reduced. There is a significant difference between the effect of the nanoparticles on the healthy cells and on the cancer cells. The effects are greater when the exposure time of the cells is increased (see 48 and 72 hours at the dose of 0.5 mg/mL on human cancer cells). The survival of the HSC cells is 20% in response to 1 mg/mL after 72 hours, whereas the survival of the SG cells (immortalised human) is 70%.

The effects on the AT84 line (mouse cancer cells) are highly significant at a concentration of 1 mg/mL (p<0.001) on and after 24 hours and are confirmed after 48 and 72 hours. This allows the conclusion that it is the cancerogenicity of the cells which is responsible for the greater sensitivity to SiC nanoparticles.

The results obtained on healthy and cancerous cell lines in culture show that the SiC nanoparticles specifically reduce the number of cancer cells, whereas the healthy cells are very significantly less affected by the SiC nanoparticles.

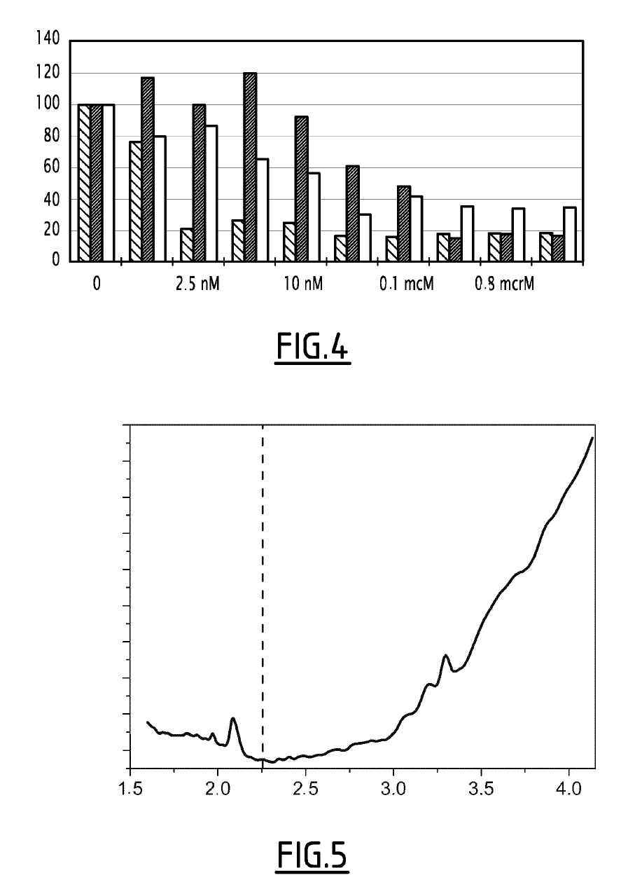

The comparison of the effects of the nanoparticles with an anticancer molecule (paclitaxel) shows that the inhibition percentages of cell survival are of the same order (FIG. 4).

It is to be pointed out that the treatment with the nanoparticles only lasted 18 hours and that the cells were then rinsed, whereas the anticancer drugs were applied to the cell cultures during the 72 hours of the assay.

The difference in the effect of the nanoparticles on the cancer cells and non-cancer cells evidences a selective effect of the SiC nanoparticles on the inhibition of cancer cells.

### Example 2

### Preparation of Nanoparticles by Laser Ablation and Properties of the Nanoparticles Thus Obtained

SiC nanoparticles were prepared using the laser ablation method following the protocol described in the following publications: A. V. Kabashin et al., J. Photochem. Photobiol. A (2006), 182 330, A. V. Kabashin et al. (2003), J. Appl. Phys., 94, 7941 and S. Barcikowski et al. (2007) Appl. Phys. A, 87, 47.

Nanoparticles of small size were thus obtained, namely of less than 10 nm.

According to FIG. 5, it is ascertained that the absorption threshold of all the nanoparticles obtained lies in the region of 3 eV, indicating that most nanoparticles have a size of less than 5.4 nm.

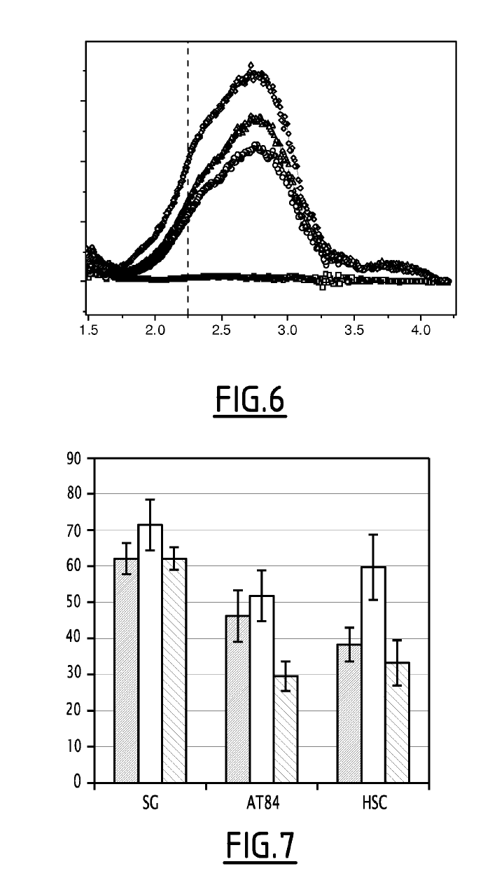

In addition, according to FIG. 6, the high photoluminescence intensities which correspond to energies higher than 2.25 eV confirm that the sizes of the nanoparticles are smaller than 5.4 nm (Bohr diameter of the exciton in 3C—SiC).

### Example 3

### Combination of Nanoparticles According to the Invention with Anticancer Agents

Assays were carried out to compare the effects of SiC nanoparticles on cell survival with the effects of cisplatin (control anticancer drug). Other assays were also carried out to compare the effects on cell survival of the combination of SiC nanoparticles and cisplatin, with the effects of cisplatin alone and the effects of the SiC nanoparticles alone.

The results are given in FIG. 7.

Cell survival was measured using a colorimetric method with 3-(4,5-diemthylthiazol-2-yl)-2,5-diphenyltetrazolium bromide (MTT) such as described above in Example 1.

Cell survival was measured after exposure to the SiC nanoparticles of Example 1 (0.5 mg/mL), to cisplatin (1.5 μM) and to the mixture of SiC nanoparticles (0.5 mg/mL) and cisplatin (1.5 μM).

The cells were cultured on 96-well plates in the presence of SiC nanoparticles.

Cell viability was measured after 72 hours. The cell cultures were then used to carry out the MTT assay.

Cell viability was measured by the difference in optical density as described in Example 1.

The indicated results are normalised with respect to the control (non-treated cells).

According to FIG. 7, it is ascertained as already indicated in Example 1, that the SiC nanoparticles of the invention have a lesser effect on the survival of healthy cells (SG line) than on the cancer cells (lines AT84 and HSC).

For the cancer cell lines, the addition of SiC nanoparticles allows an increase in the effect of cisplatin on cell survival, which is an indication of synergy.

It is observed that the survival of cells placed in culture with the cocktail of cisplatin+SiC nanoparticles is lower than that of cells placed in culture with cisplatin alone.

1.-11. (canceled)

12. A method for treating cancer, comprising administering a pharmaceutically acceptable amount of SiC nanoparticles to a patient in need of treatment for cancer, wherein said nanoparticles have a size of less than 100 nm.

13. The method of claim 12, wherein the SiC nanoparticles have a size from 0.5 nm to 100 nm.

14. The method of claim 12, wherein the SiC nanoparticles have a size of less than 10 nm.

15. The method of claim 12, wherein the SiC nanoparticles have a size of less than 7 nm.

16. The method of claim 12, wherein the SiC nanoparticles have a size of less than 5 nm.

17. The method of claim 12, wherein the SiC nanoparticles are in the form of a suspension in an aqueous physiological buffer.

18. The method of claim 17, wherein the SiC nanoparticles are present at a concentration from 0 to 20 g/L.

19. The method of claim 17, wherein the SiC nanoparticles are at a concentration from 0.5 g/L to 2 g/L.

20. The method of claim 12, wherein the SiC nanoparticles are administered via intravenous route, via local injection, via cutaneous route or via oral route.

21. The method of claim 12, wherein the SiC nanoparticles are obtained by laser ablation of a SiC substrate, a laser pyrolsyis method, or a SiC substrate etching method, wherein the etching is by electrochemical attack of the SiC substrate.

22. The method of claim 1, for treating advanced forms of cancer with metastasis.

23. The method of claim 1, wherein the SiC nanoparticles are chemically functionalized.

24. The method of claim 1, wherein an anticancer drug is grafted on the surface of the SiC nanoparticles.

25. The method of claim 1, wherein the SiC nanoparticles are administered in combination with an anticancer drug.
